(19) United States
(12) Patent Application Publication
(10) Pub. No.: US 2013/0190613 A1
(43) Pub. Date: Jul. 25, 2013

(54) Real-Time Biomechanical Dosimetry using Optical Coherence Elastography

(71) Applicant: The Board of Trustees of the University of Illinois, Urbana, IL (US)

(72) Inventors: Stephen A. Boppart, Champaign, IL (US); Jongsik Kim, Champaign, IL (US); Vasilica Crecea, Urbana, IL (US)

(73) Assignee: The Board of Trustees of the University of Illinois, Urbana, IL (US)

(21) Appl. No.: 13/739,075

(22) Filed: Jan. 11, 2013

Related U.S. Application Data
(60) Provisional application No. 61/588,884, filed on Jan. 20, 2012.

(51) Int. Cl. A61B 5/00; A61B 18/12; A61M 13/00; A61B 18/02; A61N 2/00; A61N 5/06; A61N 5/10; A61N 7/00

(52) U.S. Cl. CPC A61B 5/0066; A61B 5/4839; A61N 5/1067; A61B 18/12; A61N 7/00; A61B 18/02; A61N 2/00; A61N 5/06; A61M 13/003
USPC 600/427

(57) ABSTRACT

Methods for quantifying, adjusting or terminating a dose of a therapeutic intervention applied to tissue of a patient. A therapeutic intervention of a specified intensity is applied to a region of interest of the tissue. The tissue is mechanically excited, typically concurrently with the therapeutic intervention, and the region of interest is scanned optically or ultrasonically at the same time. An interference signal is acquired by coherently detecting post-interaction illumination arising in the region of interest by interfering the post-interaction illumination with a reference beam derived from the identical source as that of the scanning. A phase and/or amplitude of response of the tissue relative to the mechanical excitation based on the interference signal. A spatially resolved measure of a property of the region of interest is derived based on the phase of response, allowing for adjustment or termination of the therapeutic intervention.

The present Application claims the priority of U.S. Provisional Application Ser. No. 61/588,884, filed Jan. 20, 2012, and incorporated herein by reference.

### TECHNICAL FIELD

The present invention relates to apparatus and methods for spatially mapping and governing a delivered therapeutic dose of energy, and, more particularly, to mapping and controlling delivered dose by means of elastographic imaging, such as optical coherence elastography.

### BACKGROUND ART

“Interventional radiology” (IR) refers to the use of imaging technology to guide any therapeutic intervention or treatment of disease. IR has been practiced since the 1960s, and has opened the door to a multitude of life-saving minimally-invasive interventions. In IR, a surgeon or other practitioner is aided by one or more imaging modalities that supplement the surgeon's own vision, whether by means of a catheter inserted into a patient's tissue, or via a concurrent X-ray angiographic monitor, for example. Many forms of intervention and imaging fall within the rubric of IR, and many procedures are performed routinely that employ its techniques.

Among the large number of methods or energy sources (e.g., x-ray/gamma radiation, radio frequency (RF) ablation, ultrasound ablation, cryoablation, magnetic hyperthermia, etc.) that are used to treat diseases such as cancer across all the body systems, some act either through a thermal effect or are accompanied by a concomitant thermal effect.

### DEFINITIONS

As used herein and in any appended claims, the term “thermal” is used in the broad sense in which it is used in the physical sciences, namely, “relating to the internal energy of a medium due to the kinetic energy of its elementary particles, atoms, or molecules.” Thus, a region of a medium need not be in thermal equilibrium, and need not be characterized by an equilibrium temperature, in order for it to be characterized in thermal terms. The local temperature of a voxel of a sample may be in disequilibrium with the surrounding lattice, and may be characterized by an instantaneous temperature that differs from that of surrounding matter. Regions of an inhomogeneous medium may be characterized by distinct local temperatures and local thermal disequilibrium induced by magnetic anisotropies, thermal radiation, or for any of a variety of other reasons.

As used herein, and in any appended claims, the term “therapeutic thermal effect” shall refer to an effect having a thermal aspect or characteristic that is induced for purposes of treating a disease or biological anomaly.

To concretize the general concept of a therapeutic thermal effect, one example of the application of energy for therapeutic ends is magnetic hyperthermia in conjunction with magnetically responsive materials (MRMs) such as magnetic nanoparticles, magnetic microspheres, etc. Magnetic hyperthermia is currently used as an experimental cancer therapy and consists of heating a tumor region to elevate temperatures of the tumor region to temperatures above body temperature for an extended period of time. When the MRMs are exposed to an alternating magnetic field, they produce heat due to electromagnetical excitation (e.g., Eddy current, hysteresis loss, Brownian relaxation, Néel relaxation (in which the internal magnetization of the MRMs reverses direction), etc.). Typically, the alternating magnetic field has an amplitude of at least 1.5 mT and a frequency of at least 50 kHz. If the MRMs are functionalized to target cancer cells, the tumor temperature can be raised above 45° C. The temperature increase leads to thermal inactivation of cell regulatory and growth processes, with resulting widespread cell necrosis and coagulation. In addition, the thermal treatment of the tumor improves the efficacy of other treatments (e.g., radiation, chemotherapy, or immunotherapy).

Another example of an interventional radiology procedure is image-guided high intensity focused ultrasound (HIFU) ablation of tumors. The treatment concept is very similar to magnetic hyperthermia except that the heat source is focused ultrasound. In general, these interventional procedures heat or freeze (ablate) tissues in an effort to locally and selectively kill diseased tissue.

Virtually all interventional treatments still suffer from significant inefficiencies due to lack of treatment dose control. For example, the current dosimetry technique typically employed during hyperthermia treatments involves the use of a thermal probe to monitor the temperature increase of tissue due to thermal dissipation. In the case of MRMs hyperthermia, the thermal probe method is not sensitive enough to monitor the dose of hyperthermia treatment because the water content of tissue is generally 90% of the tissue volume, and this water content becomes a large heat sink compared to the MRMs heat dissipation. In other words, healthy tissue damage has already occurred by the time that a thermal probe detects tissue temperatures above 45° C.

Currently, virtually all dosimetry techniques rely on temperature probes or the use of clinical imaging modalities such as magnetic resonance imaging (MRI) or computed tomography (CT), to visualize changes in image contrast that are indicative of a temperature or structural change in the tissue being treated.

Optical coherence elastography (OCE) is now a well-established modality for imaging the mechanical properties of tissue. Tissue is driven mechanically, exciting phonons within the medium. Various excitation mechanisms have been described, such as acoustomotive OCE (AM-OCE) and magnetomotive OCE (MM-OCE). In particular, OCE, as described, for example, in Liang et al., Dynamic spectral-domain optical coherence elastography for tissue characterization, Opt. Express, vol. 18, pp. 14183-90 (2010), can distinguish regions of distinct elastic moduli, and, by implication, regions of tumorous and non-tumorous tissue. The use of OCE for resonant acoustic spectroscopy is described by Oldenburg et al., “Resonant acoustic spectroscopy of soft tissues using embedded magnetomotive nanotransducers and optical coherence tomography,” Phys. Med. Biol., vol. 55, pp. 1189-1201 (2010), which is incorporated herein by reference. A review of prior art OCE techniques may be found in Liang et al., Dynamic Optical Coherence Elastography: A Review, J. Innovative Opt. Health Sciences, vol. 3, pp. 221-33 (2010), which is incorporated herein by reference. The use of OCE for characterizing human skin is described in Liang et al., Biomechanical properties of in vivo human skin from dynamic optical coherence elastography, IEEE Trans. Biomed. Eng., vol. 57, pp. 953-59 (2010), also incorporated herein by reference.

It is well-established that tissue heating (hyperthermia) or cooling (hypothermia) will have a reversible or irreversible change in the biomechanical and/or bio-optical properties of the tissue. Once a change becomes irreversible, unintended damage may have been caused to the tissue. A dosimetry technique that would allow treatments to be monitored based on real-time measurements of tissue biomechanics would, thus, be of immense clinical impact.

### SUMMARY OF EMBODIMENTS OF THE INVENTION

In accordance with embodiments of the invention, methods are provided for quantifying a dose of a therapeutic intervention applied to tissue of a human patient. In accordance with one embodiment of the invention, such a method has steps of:

a. applying a therapeutic intervention of a specified intensity to a region of interest of tissue of a human patient;

b. mechanically exciting the tissue;

c. scanning the region of interest with optical illumination derived from an optical source, concurrently with the mechanical excitation;

d. acquiring an interference signal by coherently detecting post-interaction optical illumination arising in the region of interest by interfering the post-interaction optical illumination with a reference beam derived from the identical optical source;

e. measuring at least one of a phase and an amplitude of response of the tissue relative to the mechanical excitation based on the interference signal;

f. deriving a spatially resolved measure of a property of the region of interest based on the phase of response; and

g. terminating the therapeutic intervention based at least upon the spatially resolved measure relative to a specified criterion.

In accordance with other embodiments of the present invention, the intensity of therapeutic intervention may be modulated based on the spatially resolved measure of the property. A resonant frequency of response of the medium may also be derived. The property of the region of interest, of which a spatially resolved measure is derived, may include a mechanical property or an optical property. Examples of a mechanical property include at least one of strain, stress, strength, Young's modulus, creep, and viscosity. Examples of an optical property include at least one of refractive index, opacity, backscattering pattern, polarization, autofluorescence.

In alternate embodiments of the invention, applying the therapeutic intervention may include at least one of x-ray radiation, gamma radiation, surgery, radio frequency ablation, ultrasound ablation, cryoablation, hypothermia, magnetic hyperthermia, and chemotherapy. The mechanical excitation may include at least one of acoustomotive and magnetomotive excitation, but is not so limited, and may also include, for example, at least one of tapping, shaking, acoustic radiation force, optical radiation force, focused air puff. Deriving a spatially resolved measure of a mechanical property of the region of interest includes applying spectral domain optical coherence elastography, or swept-source-, or full- field-optical coherence tomography, or time-domain optical coherence elastography. It may also include obtaining a three- (or four-) dimensional image of the region of interest, and deriving a temporal feature of the region of interest.

In yet further embodiments of the present invention, treatment parameters may be adjusted in real time based on the spatially resolved measure of a property of the region of interest.

In accordance with another aspect of the present invention, a method is provided for quantifying a dose of a therapeutic intervention applied to tissue of a human patient, where the method has steps of:
a. applying a therapeutic intervention of a specified intensity to a region of interest of tissue of a human patient;
b. mechanically exciting the tissue;
c. scanning the region of interest with ultrasonic irradiation derived from an acoustic source, concurrently with the mechanical excitation;
d. acquiring an interference signal by coherently detecting post-interaction ultrasonic response arising in the region of interest by interfering the post-interaction ultrasonic response a reference beam derived from the identical acoustic source;
e. measuring at least one of a phase and an amplitude of ultrasonic response of the medium relative to the mechanical excitation based on the interference signal;
f. deriving a spatially resolved measure of a property of the region of interest based on the phase of response; and
g. modulating the therapeutic intervention based at least upon the spatially resolved measure relative to a specified criterion.

### BRIEF DESCRIPTION OF THE DRAWINGS

The foregoing features of the invention will be more readily understood by reference to the following detailed description, taken with reference to the accompanying drawings, in which:

FIG. 1A is a conceptual depiction of a system in which real-time OCE dosimetry may advantageously be applied in accordance with embodiments of the present invention; FIG. 1B is a flowchart depicting steps of real-time OCE dosimetry in accordance with embodiments of the present invention;

FIG. 2A shows an OCT system for use, in accordance with embodiments of the present invention, with the mechanical excitation modality of FIG. 2B;

FIG. 3 is a schematic depiction of a spectroscopic OCE system, for use in accordance with embodiments of the present invention;

FIG. 4A is a schematic depiction of a magnetomotive (MM) OCE system, for use in accordance with embodiments of the present invention; FIG. 4B is a transmission electron micrograph of the magnetite MRMs; FIG. 4C plots the mechanical underdamped oscillations in three polydimethylsiloxane (PDMS) tissue phantoms of distinct elastic moduli that occur when a constant magnetic field is applied at time t=0; and FIG. 4D is a plot of OCE-measured relaxation frequency as a function of the square root of elastic modulus of a sample;

FIG. 5A is a schematic depiction of a magnetic hyperthermia system, in accordance with an embodiment of the present invention; and FIG. 5B plots measured temperature increase of a PDMS tissue phantom as a function of duration of magnetic hyperthermia treatment;

FIG. 6A plots a waveform delivered to an MM-OCE coil as a function of time to oscillate MRMs; FIG. 6B is an amplitude image of an M-mode MM-OCE scan acquired at a region of interest in a tuna tissue sample; FIG. 6C is MRM-motion-induced phase change of an M-mode MM-OCE scan in accordance with an embodiment of the present invention; and FIG. 6D plots MRM-motion-induced phase change along the yellow dotted line on FIG. 6C;

FIGS. 7A-7F shows examples of M-mode MM-OCE scans and corresponding phase changes after 30 minutes of magnetic hyperthermia treatment of tuna tissue samples, all in accordance with embodiments of the present invention, and each as discussed in the Description, below; and

FIG. 8B plots the frequency and magnitude of M-mode signal at each of 5 positions within a microphage where microspheres are embedded, as indicated in FIG. 8A.

### DETAILED DESCRIPTION OF EMBODIMENTS OF THE INVENTION

### Definitions

The term “image” shall refer to any multidimensional representation, whether in tangible or otherwise perceptible form, or otherwise, whereby a value of some characteristic (amplitude, phase, etc.) is associated with each of a plurality of locations corresponding to dimensional coordinates of an object in physical space, though not necessarily mapped one-to-one thereonto. Thus, for example, the graphic display of the spatial distribution of some field, either scalar or vectorial, such as brightness or color, constitutes an image. So, also, does an array of numbers, such as a 3D holographic dataset, in a computer memory or holographic medium. Similarly, “imaging” refers to the rendering of a stated physical characteristic in terms of one or more images.

The terms “object,” “sample,” and “specimen” shall refer, interchangeably, to a tangible, non-transitory physical object, including, particularly, tissue of a live patient, capable of being rendered as an image.

The term “post-interaction optical illumination,” as used herein and in any appended claims, shall refer to light (without limitation as to the portion of the electromagnetic spectrum characterizing that light, which may be visible, infrared, ultraviolet, etc.) that has traversed a specimen (in transmission) or that has been scattered by the specimen.

The term “thermal excitation,” as used herein and in any appended claims, shall refer to any mechanism which varies (up or down) the local mean kinetic energy of at least a portion of a sample, and shall include, for example, and without limitation, x-ray/gamma radiation, radio frequency (RF) ablation, ultrasound ablation, cryoablation, magnetic hyperthermia, etc. When such thermal excitation is applied in order to treat a disease or biological anomaly, it may be referred to herein as a “therapeutic thermal excitation.”

A “therapeutic intervention” shall include any intervention, by any modality, into tissue of a patient with the objective of treating a disease or biological anomaly, whether by introduction of a substance (as in chemotherapy, for example), or of energy, or, equally, by extraction of a substance or of energy. Insofar as any of the foregoing modes of therapeutic intervention result in modification of mechanical or optical properties of tissue, the regulation of their dose is within the scope of the present invention.

The “dose” of a therapeutic intervention shall refer to the cumulative intensity (by any measure adopted by practitioners of a particular art) of the therapeutic intervention over the course of a specified interval of time, such as, for example, from the onset of a procedure to the current time.

The term “mechanical excitation” shall refer to inducing a mechanical perturbation within a medium, in any manner, or exciting a longitudinal wave (phonon) of any sort, whether by pressing on the tissue, using a mechanical vibrator, mechanically moving a needle, using a piezoelectric device, or any other transducer, for driving the medium for inducing movement or vibrations, acoustomotively, such as with ultrasound, for example, or magnetomotively, photoacoustically, or in any other manner, without limitation. Other methods of mechanical excitation included within the scope of the present invention, provided, again, as examples and without limitation, include tapping, shaking, acoustic radiation force, optical radiation force, and focused air puff.

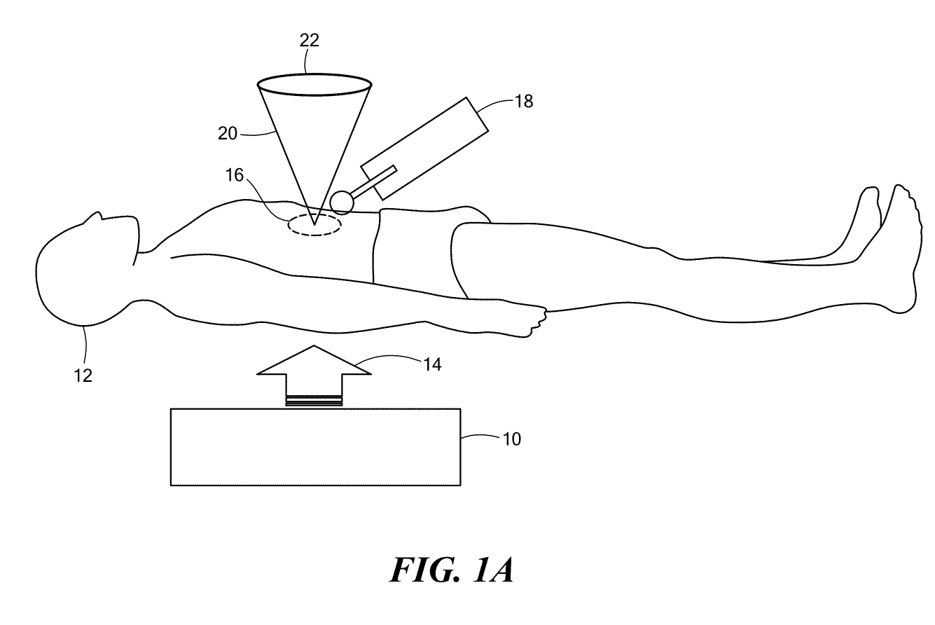

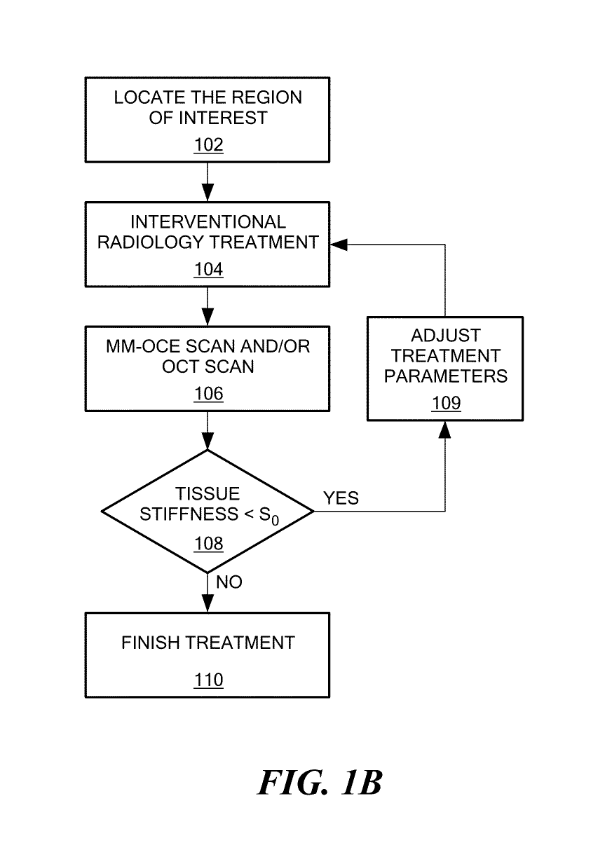

In accordance with embodiments of the present invention, methods are taught for monitoring the dose of interventional treatments in real time, as now described with reference to FIGS. 1A and 1B. Methods in accordance with the present invention may advantageously measure changes, on a microscopic scale, and with high sensitivity, in the properties of tissue. Tissue properties that may be measured for dosimetric purposes, in accordance with the present invention, include mechanical properties such as strain, stress, strength, Young's modulus, creep, viscosity, speed of sound, etc., although the foregoing are provided by way of example only, and without limitation. Additionally, or alternatively, optical properties may be measured, such as refractive index, opacity, backscattering pattern, polarization, autofluorescence, etc., again, by way of example, and without limitation.

FIG. 1A schematically depicts a system for application of methods of the present invention for purposes of real-time dosimetry. A therapeutic device 10 exposes human patient 12 to an interventional radiology treatment 14 of any of the modalities (electromagnetic, radiative, surgical, chemical, thermal, etc.) discussed above. During the course of application of the treatment 14, a region of interest (RoI) 16 of the patient is mechanically excited by means of actuator 18, which represents any of a number of possible excitation modalities, all as discussed in the present description. Treatment 14 and excitation by actuator 18 are shown in FIG. 1B as occurring on opposite sides of patient 12 solely for ease of depiction, and are, more typically, applied from the same side of the patient. The response of RoI 16 to mechanical excitation is monitored by elastographic imaging. In the embodiment depicted schematically in FIG. 1A, the elastographic imaging is performed by directing an OCT scanning beam 20 via focusing lens 22, although it is to be understood that these elements are merely exemplary of various elastographic imaging modalities that are encompassed within the scope of the invention as claimed.

In embodiments depicted in FIG. 1B, a region of interest (RoI) is located, in step 102, within tissue of a patient, by a surgeon, or by other practitioner, or using automated techniques. An interventional radiology treatment is applied 104, which interventional radiology treatment may include any modality that has a direct, or indirect, thermal effect, in the sense defined above. The region of interest is scanned 106 during the course of the interventional radiology treatment, or during intermissions in the application of the interventional radiology treatment, using an OCE scan or an MM-OCE scan, or both, or another imaging modality such as ultrasound. From the OCE or MM-OCE scan, one or more tissue properties are determined locally 108, by virtue of measured amplitude or phase, or both, in response to an applied mechanical excitation, as a function of position within the tissue of the patient. Imaging may be performed in two dimensions or in three dimensions, as described in Kennedy, et al., In vivo three-dimensional optical coherence elastography, Opt. Express, vol. 19, pp. 6623-34 (2011), which is incorporated herein by reference. In FIG. 1B, the quantitative determination of tissue stiffness S is depicted, solely by way of example. If one or more tissue properties, determined in accordance with the present invention, meet specified criteria, the interventional radiology treatment 104 is continued, or restarted, as the case may be. The interventional radiology treatment may also be adjusted or modulated (step 109) in real time, based on the determination of a tissue property in step 108. It is to be understood that, within the scope of the present invention, the intensity of treatment 104 may also be modulated on the basis of the mechanical characteristics of the tissue, determined in accordance with embodiments of the present invention. If other specified criteria with respect to determined mechanical characteristics of the tissue are met, the interventional radiology treatment is terminated 110. A criterion for termination of treatment might be a tissue stiffness reaching a value S₀, as depicted in FIG. 1, by way of example. In that case, tissue stiffness S₀ is the targeted tissue stiffness to achieve the efficacy of interventional treatment in clinic. Other dosimetry metrics that may be applied include spectroscopic content and birefringence of an OCE signal.

It is to be understood that any reference to OCE herein should be understood as encompassing any dimensionality of optical coherence imaging, including optical coherence tomography (OCT), and also as encompassing all modalities of optical coherence imaging such as spectral OCT, full-field OCE, polarization-sensitive OCE (or OCT), etc., all provided as examples and without limitation of the scope of the present invention. Moreover, time may be included as one of the dimensions of the imaging, thus temporal changes in measured properties and rates of change may be taken into account.

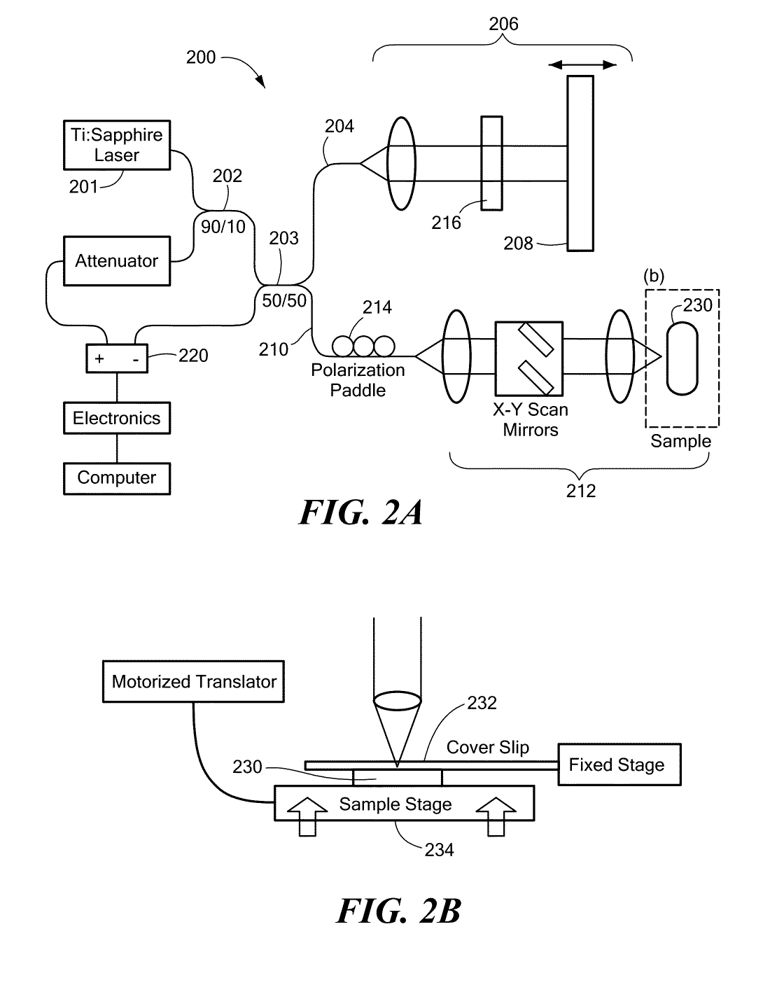

Imaging modalities for determining mechanical properties of tissue with OCE and MM-OCE technology are described, now, with reference to FIGS. 2-4. First, an OCT/OCE system is described with reference to FIG. 2A-2B, and is more fully described in Ko et al., Optical coherence elastography of engineered and developing tissue, Tissue Eng., vol. 12, pp. 63-73 (2006), which is incorporated herein by reference. Light leaving optical source 201, which may be an Nd:YVO₄-pumped titanium:sapphire laser, for example, is first split 10/90 and then 50/50 by fiber couplers 202 and 203. One fiber 204 delivers approximately half of the light to a reference arm 206 containing a linearly translating mirror 208, while another fiber 210 directs approximately half of the light to a sample arm 212 of interferometer 200. Polarization paddles 214 and dispersion matching glass 216 in the sample arm and the reference arm, respectively, help maximize the interference signal. Dual-balanced detection is implemented in detector 220 to decrease background noise.

A sample 230 under study may be confined between a fixed upper stage 232 and a sample stage 234. Step-like static compressions may be introduced by a computer-controlled translation stage 234 to demonstrate the effects of changing mechanical properties of a sample.

Tissue mechanical properties change when tissue is exposed to high or low temperatures, and the alteration of these properties is related to thermal or cryogenic injury. For example, in common experience, the stiffness of meat is increased at elevated temperature (cooking). Similarly, tissue stiffness change due to the interventional radiological treatment is highly correlated to the treatment dose. The OCE and MM-OCE technologies enable real-time dosimetry in addition to providing structural information from the optical coherence tomography (OCT) scan by virtue of the correlation of resonance frequency of tissue with its stiffness. As in any harmonic system, the resonant frequency scales with the square root of the amplitude of the restoring force, and thus, in a solid, with the square root of its stiffness. Ko et al. (2006) demonstrated the measurement of this behavior, and its special resolution in an excited sample, using OCE.

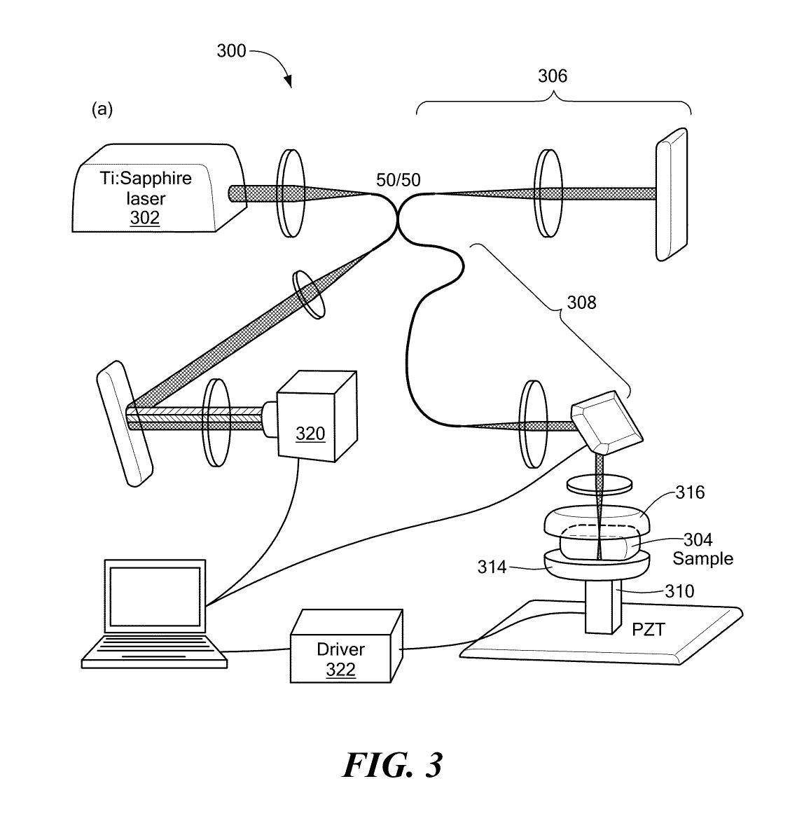

One example of an OCE that may be employed in accordance with embodiments of the present invention is a spectroscopic OCE system 300 now described with reference to FIG. 3. Spectroscopic OCE system 300 is based on spectral-domain OCT (SD-OCT) technology, and is described in detail in Adie, et al., Spectroscopic optical coherence elastography, Opt. Express, vol. 18, pp. 25519-34 (2010), which is incorporated herein by reference. A broadband source 302 provides for illumination of a sample 304 and for a reference path 306. A Nd:YVO₄-pumped titanium:sapphire laser may serve as broadband source 302, providing a center wavelength of 800 nm and a bandwidth of 100 nm. The full-width half-maximum axial and transverse resolutions of the OCE system 300 are approximately 3 μm and 13 μm, respectively. The average power incident on sample 304 is typically on the order of 10 mW. Sample arm 308 of OCE system 300 employs a piezoelectric transducer (PZT) stack 310 to sinusoidally excite the sample (distal to incident optical beam 312) in the axial direction, i.e., along the propagation direction of optical beam 312. In the exemplary embodiment depicted in FIG. 3, PZT 310 was driven with a maximum displacement of 4.5 μm at vibration frequencies within the range DC to 1 kHz. The sample was bounded from below by a coverslip 314, with approximate thickness of 125 μm, that was fixed with epoxy to the PZT rod, and from above by a round wedge prism 316 with a 2° angle fixed to form a semi-rigid upper boundary to the sample. Axial depth scans in the OCE images (depth×lateral pixel dimensions of 1024×4000), were detected using a CCD line-scan camera 320. The camera acquisition was synchronized with a transverse scanning galvanometer and the PZT excitation signal derived from driver 322.

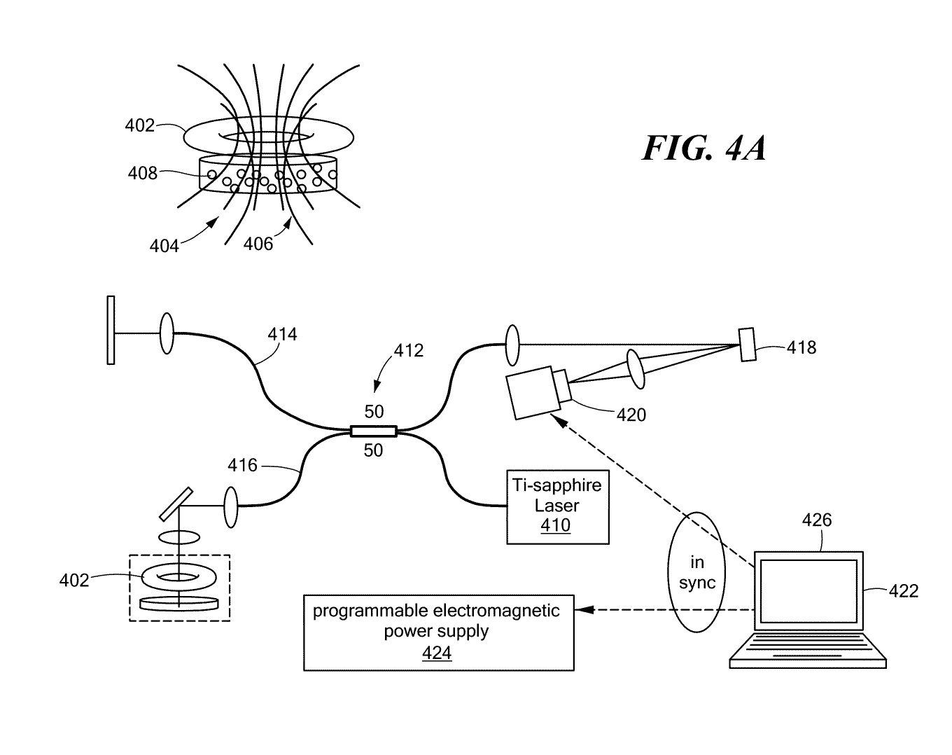

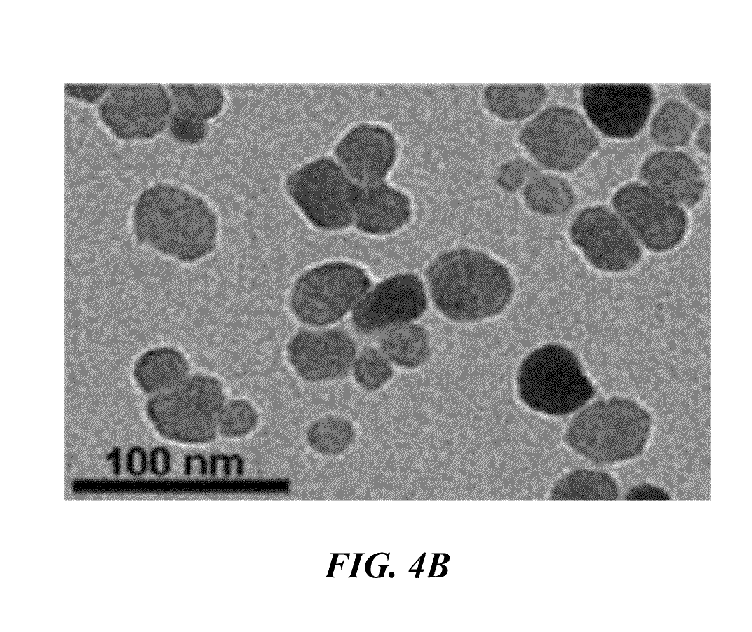

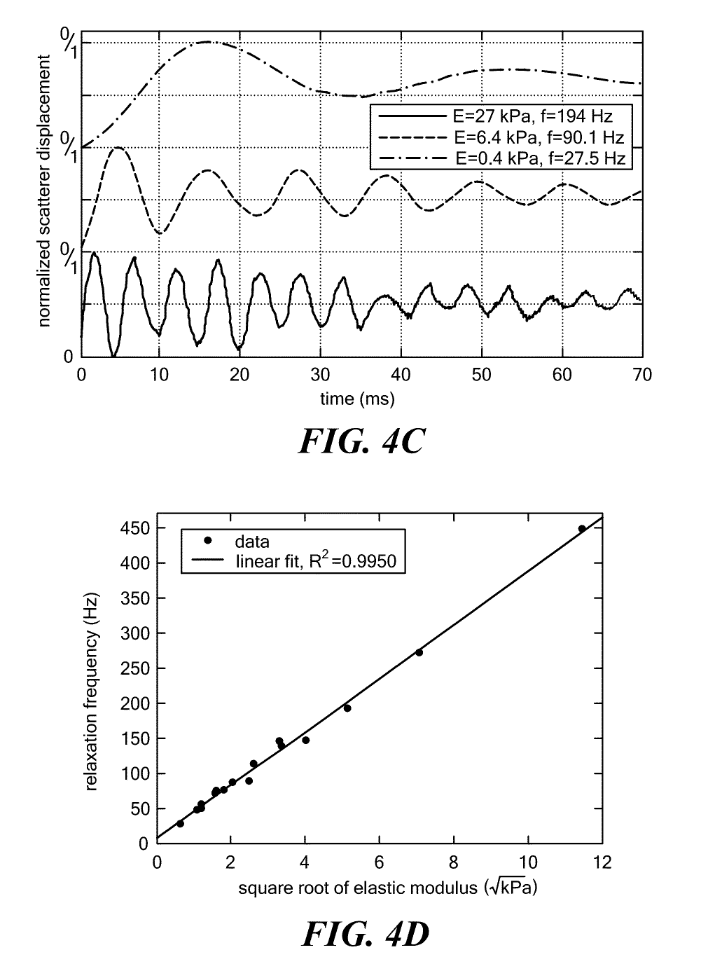

An MM-OCE setup, with MRMs, is now described with reference to FIGS. 4A-4D, with further details provided in Oldenburg, et al., Magnetomotive contrast for in vivo optical coherence tomography. Opt Express vol. 13, pp. 6597-6614 (2005), and Crecea, et al., Magnetomotive nanoparticle transducers for optical rheology of viscoelastic materials, Opt. Express, vol. 17, pp. 114-22 (2009), both of which are incorporated herein by reference. A magnetic coil 402 (shown in detail at top left) provides a magnetic field 404 that is aligned axially with an imaging beam 406. The magnetic field gradient engages the motion of MRMs 408 in the sample. FIG. 4B is from a transmission electron micrograph of the magnetite MRMs. Near-infrared light 406, constituting an OCE scanning beam, is provided by a broadband source 410 such as a titanium:sapphire laser, divided by a fiber-optic beam splitter 412 between reference arm 414 and sample arm 416 of an interferometer. The interference signal is wavelength-dispersed by a diffraction grating 418 and recorded by a charged coupled device (CCD) line array 420. The magnetic field activity is synchronized with the OCT data acquisition, by processor 422 and programmable electromagnet power supply 424. Resulting optical back-scattering data is acquired, processed, and displayed on a personal computer 426 which may also serve as processor 422. FIG. 4C shows the resonance frequency (natural frequency) of the response from the MRM motion which is measured by the MM-OCE scans. Normalized measured displacements from polydimethylsiloxane (PDMS) samples of different elastic moduli following a step (off-to-on) transition of the applied magnetic field are shown. Three samples that span a wide range of elastic moduli are shown: 0.4 kPa, 6.4 kPa, and 27 kPa. These sample moduli are characteristic of soft biological tissue, and were chosen to illustrate the natural frequencies of oscillation measured by MM-OCE. As expected, it is observed that as the stiffness of the medium increases, the natural frequency of oscillation of the response increases. Indeed, in FIG. 4D, MM-OCE-measured natural frequencies of oscillation are plotted for samples of varying elastic moduli. The natural frequency of oscillation of the viscoelastic medium depends linearly on the square root of the elastic modulus, as predicted by the Kelvin-Voigt model. The MM-OCE relaxation frequency data (vertical axis) were collected as the samples relaxed following an on-off step magnetic field transition. The elastic moduli (horizontal axis) values were measured by indentation.

Returning, now, to discussion of FIGS. 1A and 1B, interventional radiology treatments offer a unique capability for treating tumors, for example, through the use of tissue thermal or cryo-ablation. However, absent teachings of the present invention, interventional radiology is severely constrained by limitations of existing dosimetry for the interventional radiology. For example, the temperature distribution inside and outside the region of interest (i.e., cancerous region) must be known precisely as a function of the exposure time (treatment duration) in order to provide the optimum efficacy for the interventional radiology treatment and to avoid overdosing and damaging surrounding healthy tissue. Methods in accordance with embodiments of the present invention may advantageously provide real-time dosimetry based directly on the tissue biomechanical properties, and with spatial scales on the order of tens to hundreds of microns. For the first time, optimum delivery of the interventional radiology treatment may be provided.

In accordance with certain embodiments of the present invention, feedback may be provided and interventional radiological treatment may be modulated based on the changing biomechanical properties of the tissue being treated, rather than just a point temperature measurement, or using large-scale biomedical imaging modalities to image and detect contrast or structural changes in the tissue. Biomechanical changes are advantageously sampled and imaged at the micron-scale.

### Example

### Tissue Stiffness in a Magnetic Hyperthermia System

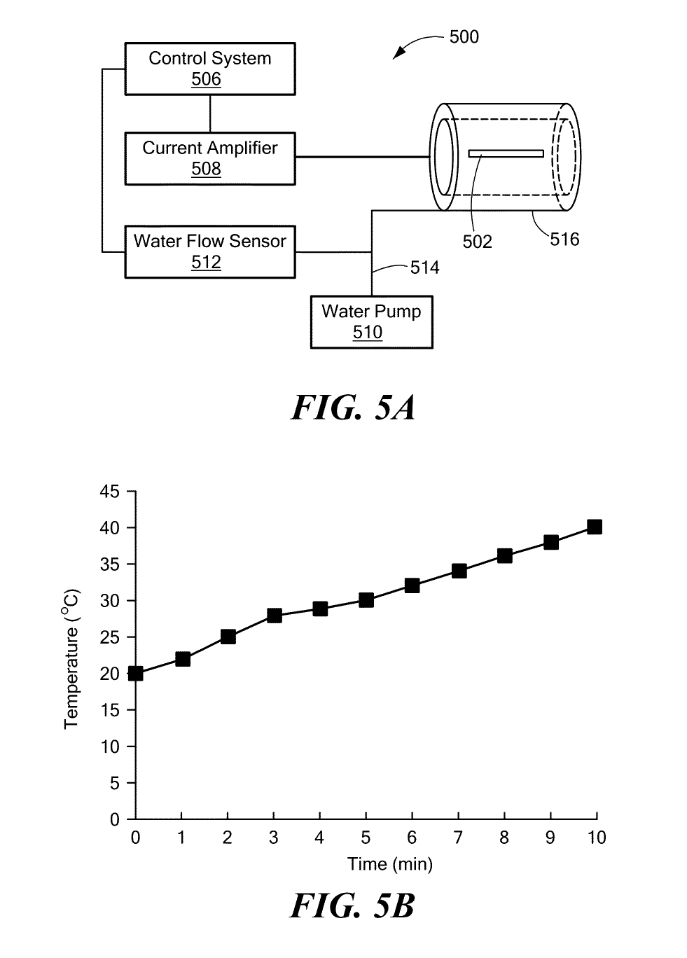

A demonstration of monitoring the tissue stiffness changes due to an interventional radiology treatment is now discussed with reference to a magnetic hyperthermia treatment system 500 for use with MRMs and depicted schematically in FIG. 5A. Typically, system 500 delivers a high magnetic field frequency (≧150 Gauss, ≧50 kHz) for a period of ≧5 min to tissue sample 502 injected with MRMs, which, in the case of the example depicted, is one of a series of PDMS tissue phantoms. Magnetomotive nanoparticle transduction is described by Crecea et al., Magnetomotive nanoparticle transducers for optical rheology of viscoelastic materials, Opt. Express, vol. 17, pp. 23114-22 (2009), incorporated herein by reference. The initial stiffness of the tissue phantoms before the magnetic hyperthermia treatment was roughly 10 kPa, which is close to the stiffness of skeletal muscle. Sample 502 is surrounded by a cylindrical induction coil 504 for providing the aforesaid magnetic field, as driven by controller 506 and current amplifier 508. A water pump 510 and attendant water flow sensor 512 provide a water bath via tubes 514 and 516. FIG. 5B shows the temperature increase of the PDMS tissue phantom, mixed with a 10 mg/mL concentration of MRMs, due to magnetic hyperthermia treatment with alternating magnetic field amplitude of 860 Gauss and a frequency of 62 kHz. The temperature of the PDMS tissue phantom reached 40° C. within 10 minutes.

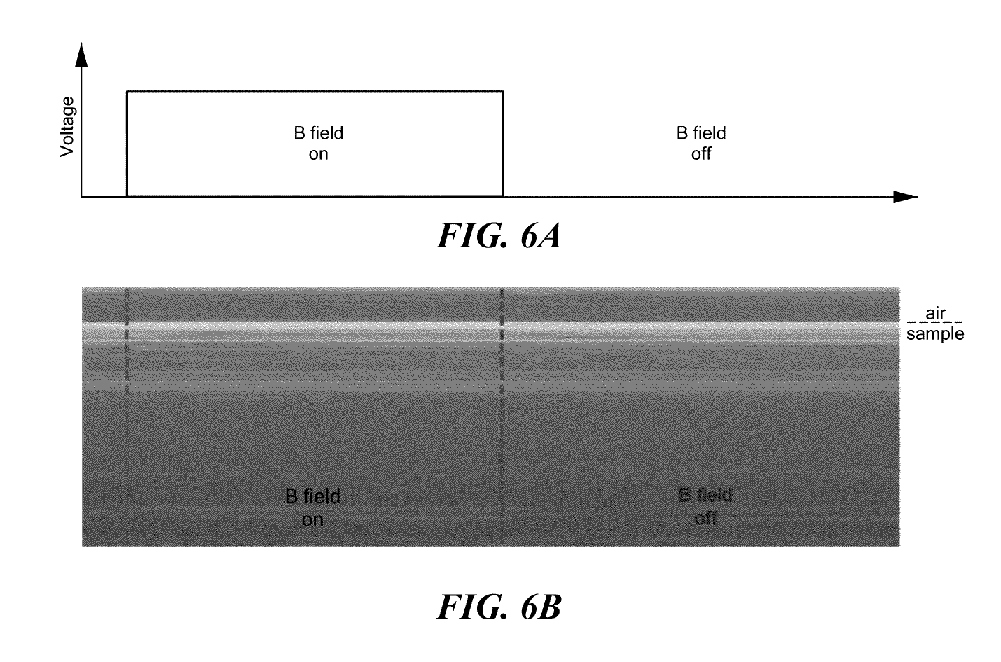

Assessment of tissue stiffness using magnetomotive optical coherence elastography (MM-OCE) process is depicted in FIGS. 6A-6D. A step function waveform, as shown in FIG. 6A, is delivered to the MM-OCE coil 504 (shown in FIG. 5A) to induce oscillations of the MRMs in tissue phantom 502. FIG. 6B shows successive A-scan lines (axial scans) with modulus of interference signal plotted as brightness as a function of depth into the sample. An M-mode MM-OCE scan (3000 A-scan lines (or time points) and 1024 pixels in depth; FIG. 6B) is acquired at the region of interest (RoI) from a tissue sample (tuna), where M-mode refers to magnetic field modulation, and is distinguished from embodiments that employ a galvo scanner for scanning the RoI. Once the M-mode MM-OCE image was acquired, the phase change (proportional to the displacement of tissue; FIG. 6C) due to MRMs motion induced by the MM-OCE coil was computed, and is plotted versus time (FIG. 6D) in an interval during which a magnetomotive excitation is applied to the sample. Finally, the natural frequency of the tissue sample response after the termination of the magnetic field (after the A-scan line of 500) was assessed, which was highly correlated with the tissue stiffness change (i.e., the stiffer the tissue, the higher the natural frequency).

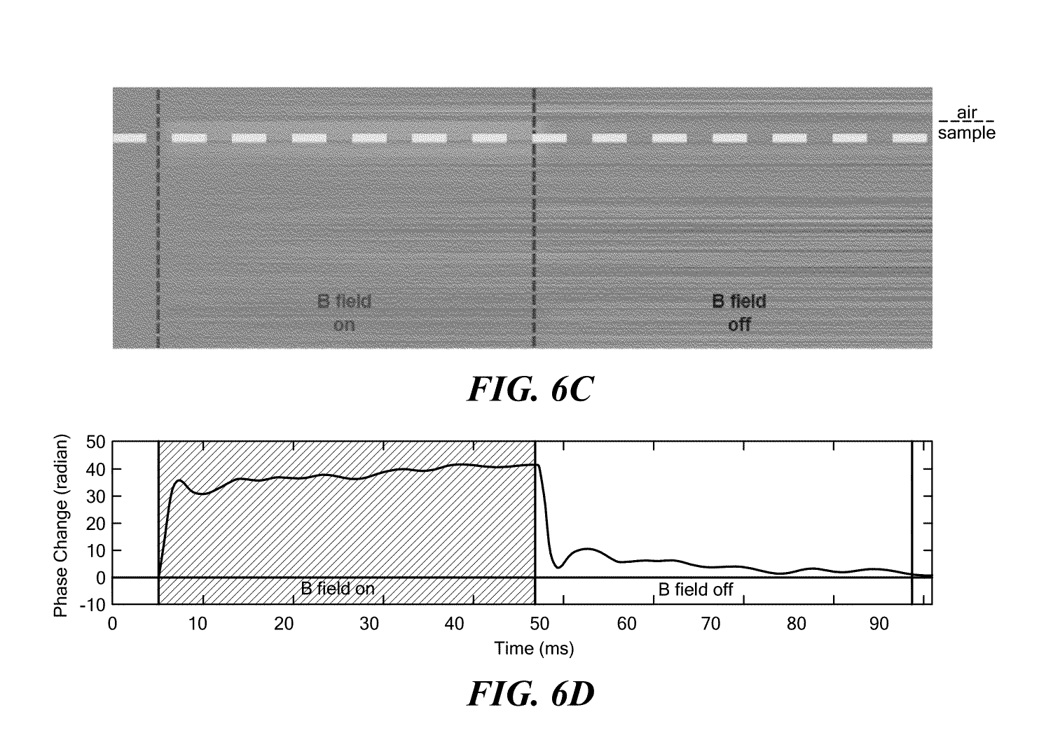

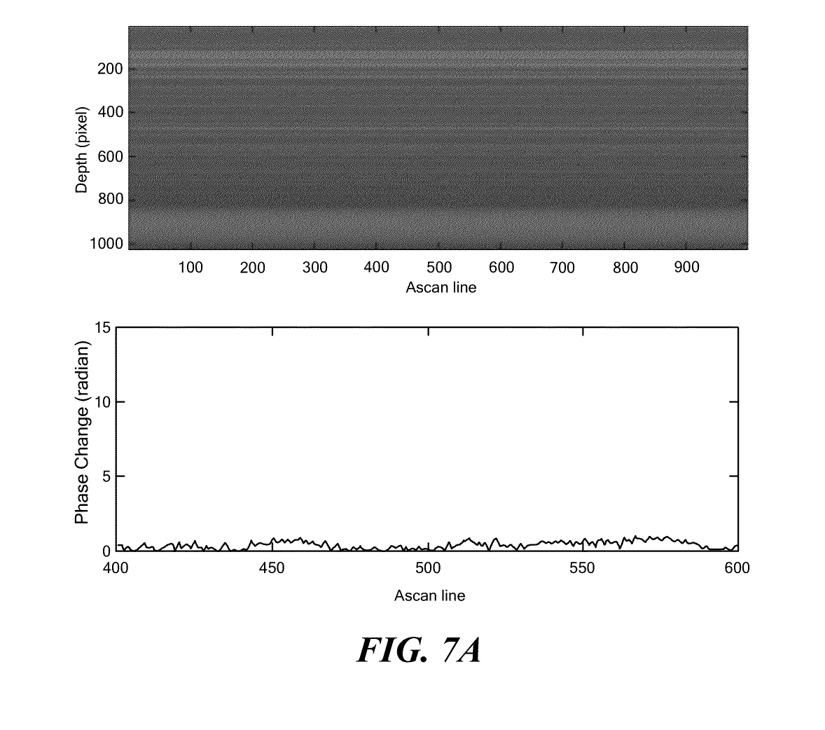

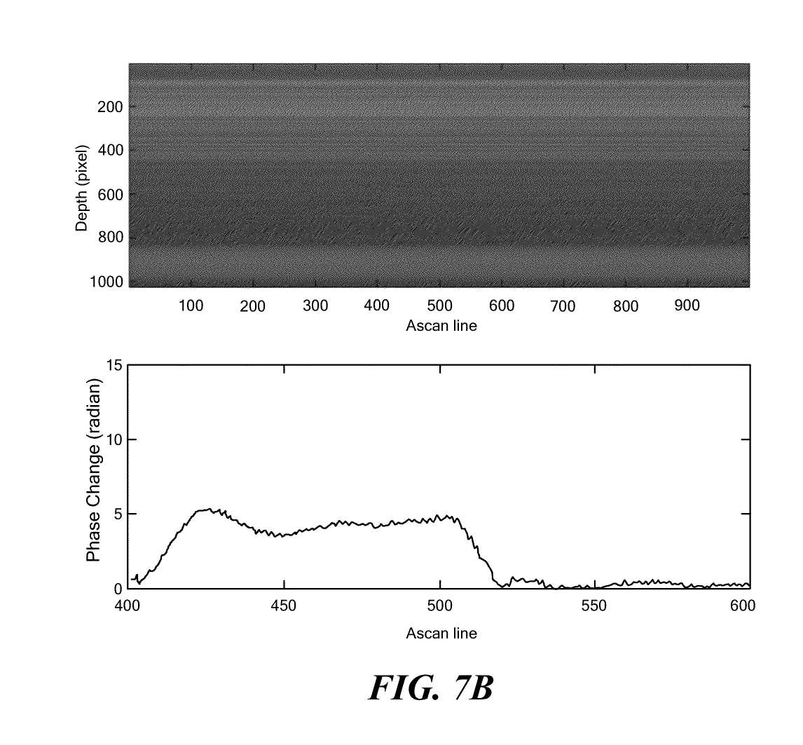

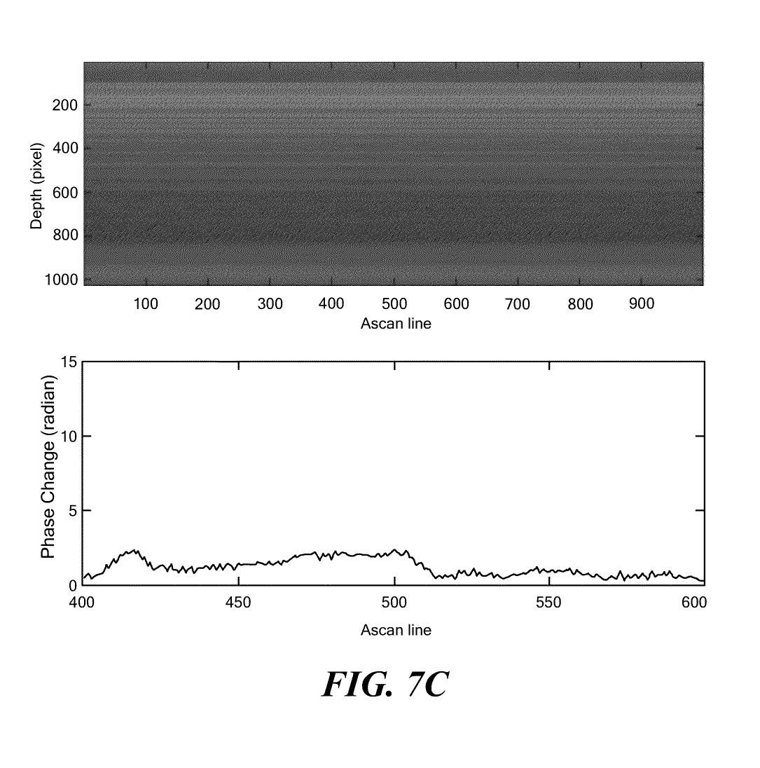

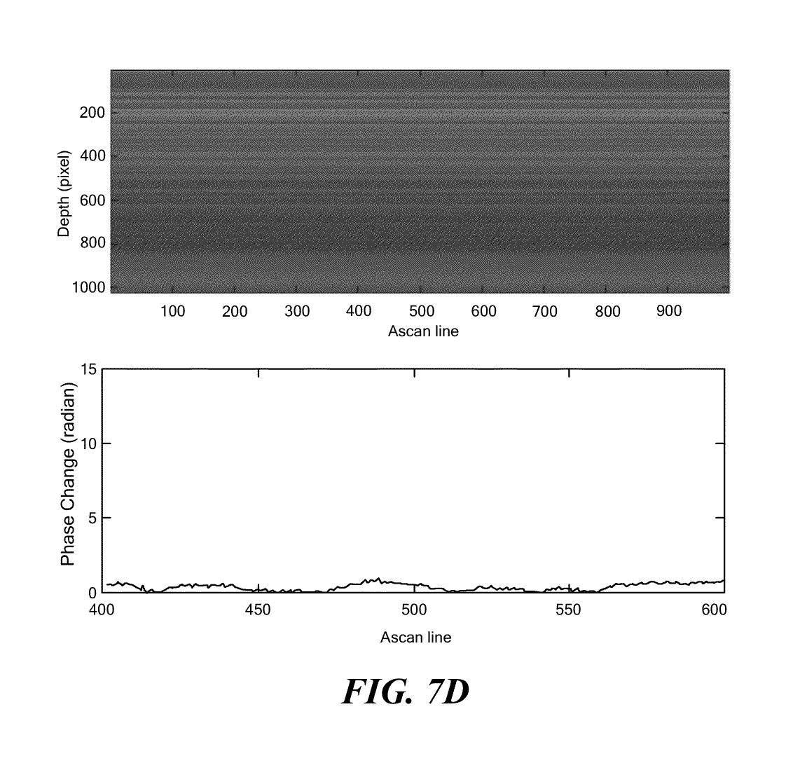

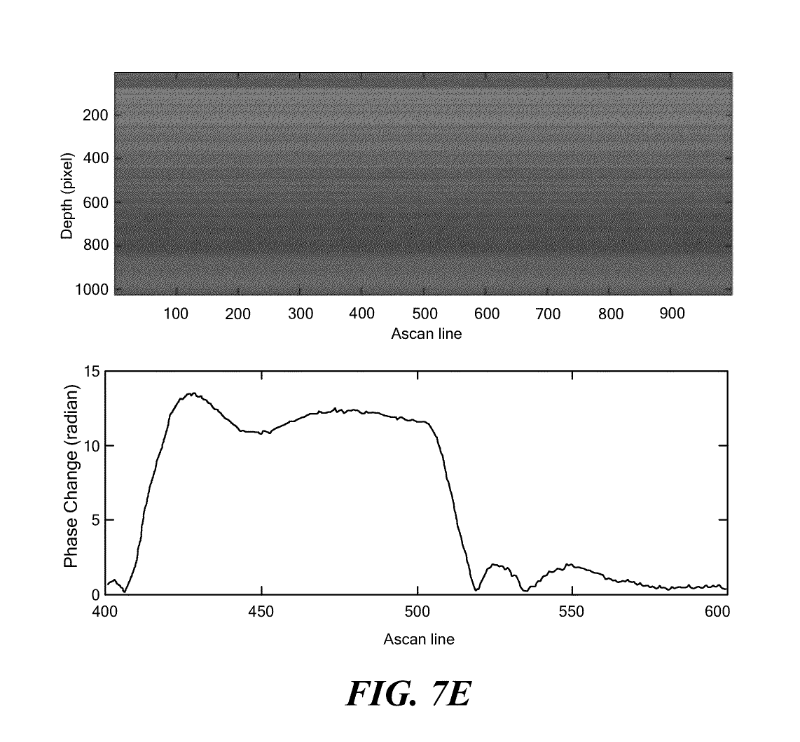

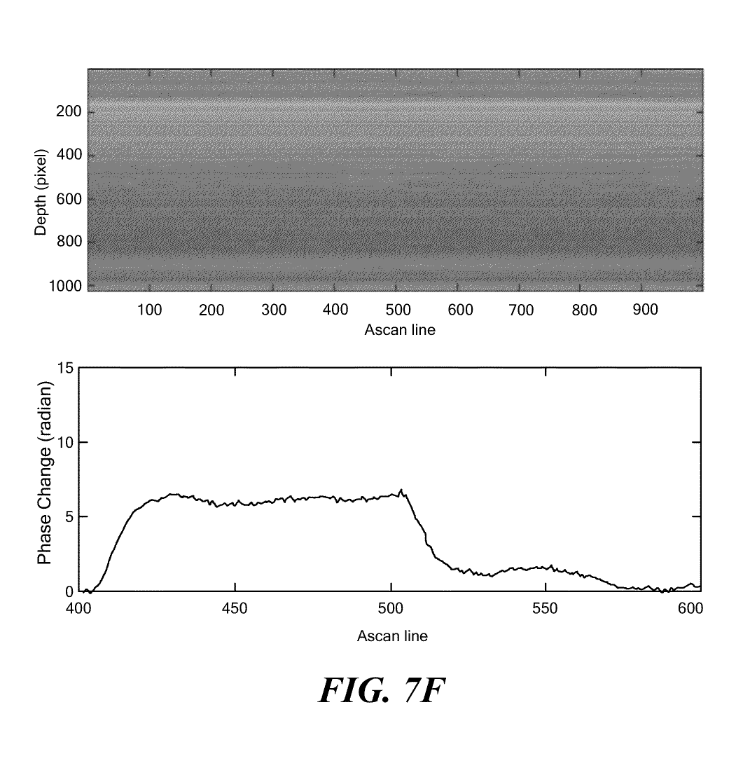

FIGS. 7A-7D depict examples of the magnetomotive optical coherence elastography (MM-OCE) scans and the corresponding phase changes associated with each at inception of the magnetic hyperthermia treatment and after 30 minutes of treatment. FIGS. 7A and 7C are controls, with no MRMs. FIG. 7B corresponds to tissue sample with 100 mg/mL concentration of MRMs at t=0 min; FIG. 7C corresponds to the control (without MRMs) at t=30 min; and FIG. 7D to tissue sample with 100 mg/mL concentration of MRMs after the magnetic hyperthermia treatment of 30 min. The control group shows no significant phase change before and after the magnetic hyperthermia treatment. However, the experimental group mixed with 100 mg/mL shows the significant change in the phase, as well as in the natural frequency before and after the magnetic hyperthermia treatment.

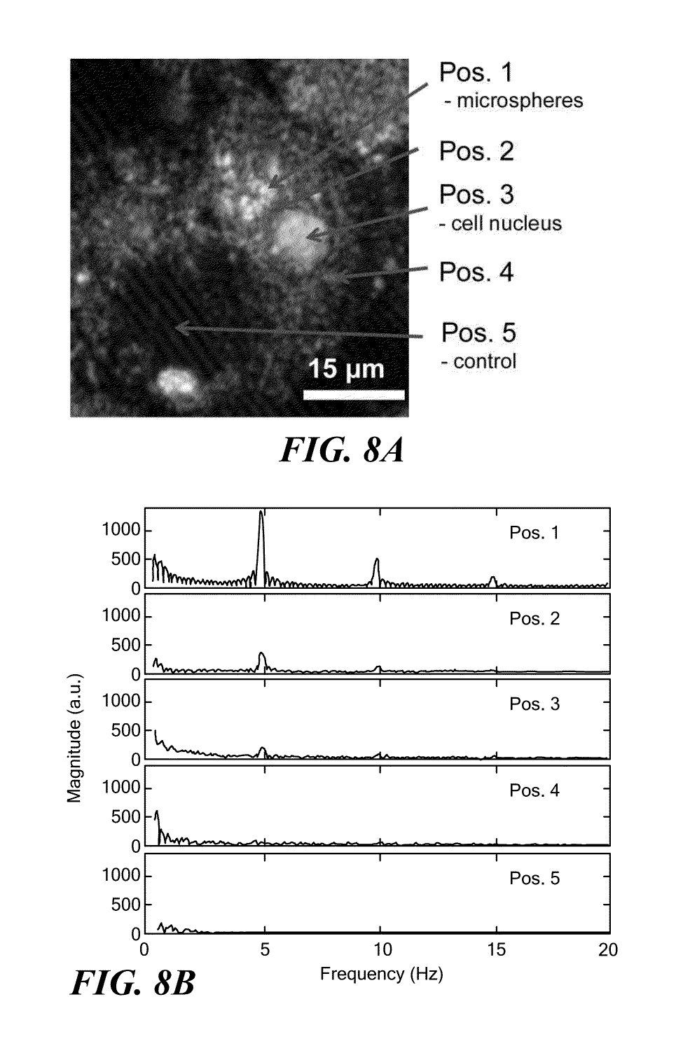

Referring now to FIGS. 8A and 8B, five positions are indicated where microspheres are embedded within a single microphage. The response amplitude of each corresponding microsphere subject to M-mode excitation in accordance with an embodiment of the present invention is plotted in FIG. 8B, illustrating resolution of techniques described herein at the cellular scale.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method for quantifying a dose of a therapeutic intervention applied to tissue of a human patient, the method comprising:
a. applying a therapeutic intervention of a specified intensity to a region of interest of tissue of a human patient;
b. mechanically exciting the tissue;
c. scanning the region of interest with optical illumination derived from an optical source, concurrently with the mechanical excitation;
d. acquiring an interference signal by coherently detecting post-interaction optical illumination arising in the region of interest by interfering the post-interaction optical illumination with a reference beam derived from the identical optical source;
e. measuring at least one of a phase and an amplitude of response of the tissue relative to the mechanical excitation based on the interference signal;
f. deriving a spatially resolved measure of a property of the region of interest based on the phase of response; and
g. terminating the therapeutic intervention based at least upon the spatially resolved measure relative to a specified criterion.

2. A method in accordance with claim 1, further comprising modulating the intensity of therapeutic intervention based on the spatially resolved measure of the property of the region of interest.

3. A method in accordance with either of claims 1 and 2, wherein the property of the region of interest is a mechanical property.

4. A method in accordance with either of claims 1 and 2, wherein the property of the region of interest is selected from the group of mechanical properties including strain, stress, strength, Young's modulus, creep, viscosity, and speed of sound.

5. A method in accordance with either of claims 1 and 2, wherein the property of the region of interest is an optical property.

6. A method in accordance with either of claims 1 and 2, wherein the property of the region of interest is selected from the group of optical properties including refractive index, opacity, backscattering pattern, polarization, autofluorescence.

7. A method in accordance with claim 1, further comprising deriving a resonant frequency of response of the medium.

8. A method in accordance with claim 1, wherein applying the therapeutic intervention includes at least one of x-ray radiation, gamma radiation, surgery, radio frequency ablation, ultrasound ablation, cryoablation, hypothermia, magnetic hyperthermia, and chemotherapy.

9. A method in accordance with claim 1, wherein mechanically exciting the tissue includes at least one of acoustomotive and magnetomotive excitation.

10. A method in accordance with claim 1, wherein mechanically exciting the tissue includes at least one of tapping, shaking, acoustic radiation force, optical radiation force, focused air puff.

11. A method in accordance with claim 1, wherein deriving a spatially resolved measure of a property of the region of interest includes applying spectral domain optical coherence elastography.

12. A method in accordance with claim 1, wherein deriving a spatially resolved measure of a property of the region of interest includes applying swept-source optical coherence elastography.

13. A method in accordance with claim 1, wherein deriving a spatially resolved measure of a property of the region of interest includes applying time domain optical coherence elastography.

14. A method in accordance with claim 1, wherein deriving a spatially resolved measure of a property of the region of interest includes applying full-field optical coherence elastography.

15. A method in accordance with claim 1, wherein deriving a spatially resolved measure of a property of the region of interest includes applying spectroscopic content or the birefringence is used as the dosimetry metric.

16. A method in accordance with claim 1, wherein deriving a spatially resolved measure of a property of the region of interest includes obtaining a three-dimensional image of the region of interest.

17. A method in accordance with claim 1, wherein deriving the spatially resolved measure of the property of the region of interest includes deriving a temporal feature.

18. A method in accordance with claim 1, further comprising adjusting treatment parameters in real time based on the spatially resolved measure of a property.

19. A method for quantifying a dose of a therapeutic intervention applied to tissue of a human patient, the method comprising:
a. applying a therapeutic intervention of a specified intensity to a region of interest of tissue of a human patient;
b. mechanically exciting the tissue;
c. scanning the region of interest with ultrasonic irradiation derived from an acoustic source, concurrently with the mechanical excitation;
d. acquiring an interference signal by coherently detecting post-interaction ultrasonic response arising in the region of interest by interfering the post-interaction ultrasonic response a reference beam derived from the identical acoustic source;
e. measuring at least one of a phase and an amplitude of ultrasonic response of the medium relative to the mechanical excitation based on the interference signal;
f. deriving a spatially resolved measure of a property of the region of interest based on the phase of response; and
g. modulating the therapeutic intervention based at least upon the spatially resolved measure relative to a specified criterion.
